(19) United States
(12) Patent Application Publication
(10) Pub. No.: US 2004/0009522 A1
(43) Pub. Date: Jan. 15, 2004

(54) Methods of selecting 5'-capped nucleic acid molecules

(75) Inventor: Ning Wu, Edmond, OK (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/448,441

(22) Filed: May 30, 2003

Related U.S. Application Data
(63) Continuation of application No. 10/241,051, filed on Sep. 10, 2002.
(60) Provisional application No. 60/323,140, filed on Sep. 12, 2001.

(51) Int. Cl. C12Q 1/68; G01N 33/53; C07H 21/04; C07H 21/02

(52) U.S. Cl. 435/6; 435/7.1; 536/25.4

(57) ABSTRACT

Methods for obtaining enriched populations of nucleic acid molecules having a 5′-cap from a population of nucleotide sequences are provided. In addition, methods of using such 5′-capped molecules also are provided, for example, methods of using a full length mRNA molecule, which is obtained by such a method and contains a 5′-cap and a polyA tail, to prepare full length cDNA molecules and libraries of such cDNA molecules.

This application claims the benefit under 35 U.S.C. 119(e) of U.S. Serial No. 60/323,140, filed Sep. 12, 2001, which is incorporated herein by reference in its entirety.

### BACKGROUND OF THE INVENTION

1. Field of the Invention

The invention relates generally to methods of obtaining enriched populations of capped RNA molecules, and more specifically to methods of obtaining full length mRNA molecules, and to isolated populations of such mRNA molecules, and cDNA and cDNA libraries prepared therefrom. The invention also relates to vectors and host cells containing such cDNA molecules.

2. Background Information

In examining the structure and physiology of an organism, tissue or cell, it is often desirable to determine its genetic content. The genetic framework of an organism is encoded in the double stranded sequence of nucleotide bases in deoxyribonucleic acid (DNA) molecules, which comprises the chromosomes present in the nuclei of somatic and germ cells of a eukaryotic organism. The genetic content of a particular segment of DNA, or gene, is only manifested upon production of the protein which the gene encodes. In order to produce a protein, the “coding” strand of DNA is transcribed by polymerase enzymes into ribonucleic acid (RNA), which, in turn, is processed into messenger RNA (mRNA).

Within a given cell, tissue or organism, there exist myriad mRNA species, each encoding a separate and specific protein. This fact provides a powerful tool to investigators interested in studying genetic expression in a tissue or cell, wherein mRNA molecules are isolated and further manipulated by various molecular biological techniques, thereby allowing the elucidation of the full functional genetic content of a cell, tissue or organism.

One common approach to the study of gene expression is the production of complementary DNA (cDNA) clones. In this technique, the mRNA molecules from an organism are isolated from an extract of the cells or tissues of the organism. Since the 3′ terminus of all eukaryotic mRNA molecules contains a string of adenosine (A) bases, and since A binds to T, the mRNA molecules can be rapidly purified from other molecules and substances in the tissue or cell extract by hybridizing RNA, including mRNA to immobilized poly T nucleotide sequences. The purified mRNA molecules than can be used as a template to make a single stranded cDNA using a reverse transcriptase (RT) enzyme, then a complementary DNA strand can be synthesized from the single stranded cDNA template using a DNA polymerase, thereby producing a double stranded cDNA molecule. The double stranded cDNA then can be conveniently manipulated, for example, by inserting it into a plasmid or a vector.

The process of isolating mRNA, preparing cDNA, inserting the cDNA into a plasmid or vector, and growing host cells containing the cDNA is termed “cDNA cloning.” If cDNAs are prepared from a number of different mRNAs, the resulting set of cDNAs is called a “cDNA library,” and is representative of the different populations of functional genetic information (genes) present in the source cell, tissue or organism. Genotypic analysis of these cDNA libraries can yield much information on the structure and function of the organisms from which they were derived.

Construction of a full length cDNA library is the prerequisite for functional analysis of different genes in a specific tissue or cell line. The key factor in full length cDNA generation is the availability of full length mRNA, which has a characteristic cap structure at its 5′ end (Furuichi and Miura, 1975), contains complete information of the coding region and the non-coding region, and terminates in a polyA tail. However, RNA molecules are extremely labile, and are readily degraded by ubiquitous enzymes (RNAses).

As such, many techniques have been developed in an attempt for the purpose of selection of full length mRNA. All of these methods focus on the manipulation of the intact 5′ end cap structure of the mRNA, including, for example, the “Oligo-capping” methods (Maruyama and Sugano, 1994) and the “Cap trapper” method (Caminci et al., 1996). However, these methods require specific enzymes or specific protein-coated magnetic beads and a number of steps to isolate the mRNA. Unfortunately, the risk of RNA degradation is increased due to the number of manipulations and longer handling time required to practice these methods. In addition, these methods require a large amount of mRNA (5 to 10 μg) to obtain the desired results. As such, the methods are impractical for isolating full length mRNA from individual smaller organs or rare tissues. Thus, a need exists for methods of isolating intact, full length mRNA. The present invention satisfies this need, and provides additional advantages.

### SUMMARY OF THE INVENTION

The present invention relates to a method of isolating or removing uncapped nucleotide sequences from a sample or reaction mixture. Such a method can be performed, for example, by contacting a sample containing one or more or a population of nucleotide sequences (preferably RNA, more preferably mRNA) with at least one agent that selectively binds uncapped nucleotide sequences, under conditions that allow the agent to selectively bind an uncapped nucleotide sequence (preferably RNA, more preferably mRNA), and removing the agent and any nucleotide sequence bound thereto from the sample. Thus, the invention allows removal or isolation of uncapped nucleic acid molecules from a sample, leaving behind capped sequences, which can be used or manipulated using standard molecular biology techniques such as for performing cDNA synthesis. The agent can be any molecule that selectively binds uncapped nucleotide sequences, including, for example, an oligonucleotide, a peptide, a carbohydrate, a lipid, a lipopolysaccharide, or a small organic molecule such as a peptidomimetic. In addition, the agent can include at least one first member of a specific binding pair, for example, two, three, four, five, six, or more first members of a specific binding pair, and can, if desired, contain two or more different first members of a binding pair, for example, a first member of a first binding pair and a first member of a second binding pair. Accordingly, the step of removing the agent and any nucleotide sequence bound thereto can be performed by contacting the sample with at least one second member of the specific binding pair.

Such a method can further include, following removing the agent and any nucleotide sequence bound thereto from the sample, a step of isolating nucleic acid molecules from the sample. Such a method provides a means for obtaining an enriched population of nucleic acid molecules comprising a 5′-cap, for example, 5′-capped RNA molecules. In one embodiment, the step of isolating nucleic acid molecules from the sample involves contacting the sample with at least one polynucleotide that can specifically hybridize to a polyadenosine nucleotide sequence, and isolating nucleic acid molecules that specifically hybridize to the polynucleotide. Such a method provides a means of obtaining an enriched population of nucleic acid molecules comprising 5′-cap and a polyadenosine nucleotide sequence, particularly, full length mRNA molecules. This isolation step performed prior to and/or after the use of the agent of the invention to isolate or remove uncapped nucleic acid molecules. Accordingly, the invention provides an enriched population of nucleic acid molecules obtained by such a method.

The present invention relates to methods for enriching for nucleic acid molecules having a 5′-cap from a sample containing nucleotide sequences, including nucleic acid molecules comprising a 5′-cap. A method of the invention can be performed, for example, by contacting a sample containing one or more or a population of nucleotide sequences and at least one agent that selectively binds at or near a free 5′-phosphate group of a nucleotide sequence, under conditions that allow the agent to selectively bind at or near a free 5′-phosphate group of a nucleotide sequence, and removing the agent and any nucleotide sequence bound thereto from the sample. The agent can be any molecule that selectively binds at or near a 5′-phosphate group of a nucleotide sequence, including, for example, an oligonucleotide, a peptide, or a small organic molecule. The agent can bind directly to the nucleotide sequence, or binding can be mediated through a chemical or enzymatic reaction. For example, that the agent can be an oligonucleotide, and selective binding of the oligonucleotide to a 5′-phosphate group of a nucleotide sequence can be mediated by a ligase.

In one embodiment, a method of the invention provides a means to obtain an enriched population of RNA molecules having a 5′-cap. In such a method, the agent can be an oligoribonucleotide, the nucleotide sequences to be removed include RNA molecules having a free 5′-phosphate, and selective binding of the agent to the nucleotide sequence is mediated by an RNA ligase. The oligonucleotide agent can have, for example, a 2′ hydroxyl group or a 3′ hydroxyl group, either of which can selectively bind a free 5′ phosphate group of the nucleotide sequences, and the sample can include an RNA ligase, for example, an E. coli 2′-5′ RNA ligase or a T4 RNA ligase, respectively, which mediates selective binding of the oligonucleotide to nucleic acid molecules having a free 5′-phosphate.

According to a method of the invention, the step of removing the agent, and any nucleotide sequences bound thereto, includes contacting the sample with at least one moiety that selectively binds the agent. The moiety can be any molecule that specifically binds the agent, for example, an antibody that can specifically bind a peptide agent, or a polynucleotide that can specifically hybridize to an oligonucleotide agent. If desired, the moiety can be coupled to a solid support, which can facilitate removal of the agent and any nucleotide sequences bound thereto.

In another embodiment, the agent includes at least one first member of a specific binding pair, which can specifically bind a second member of the specific binding pair. According to such a method, removing the agent comprises contacting the sample with at least one second member of the specific binding pair, which selectively binds the first member of the specific binding pair and, therefore, the agent and any nucleotide sequences bound thereto. The specific binding pair can be any such pair, including, for example, biotin and avidin, biotin and streptavidin, an antibody specific for an epitope and the epitope, nickel ion and polyhistidine, or glutathione and glutathione S-transferase, and can include a combination thereof. For example, an agent can be a biotinylated oligonucleotide, particularly a biotinylated oligoribonucleotide, which can contain one or more (e.g. 1, 2, 3, 4, 5, 6, 7, etc.) biotin molecules, wherein the agent can be removed by contacting the sample with avidin or streptavidin. Removing the agent containing the member of the binding pair can be performed using any method, for example, by performing a phenol/chloroform extraction method, wherein the agent and any nucleotide sequences bound thereto partition into the organic phase, and isolating the aqueous phase containing an enriched population of 5′-capped nucleic acid molecules; or by contacting the sample with at least one second member of the specific binding pair, for example, avidin or streptavidin, which is coupled to a solid support.

The present invention also relates to methods of using such an enriched population of 5′-capped nucleic acid molecules. For example, a method of the invention can further include contacting the enriched population of nucleic acid molecules comprising a 5′-cap with at least one polynucleotide that selectively hybridizes to a polyadenosine nucleotide sequence of a nucleic acid molecule, and isolating nucleic acid molecules that selectively hybridize to the polynucleotide. Such a method provides a means to obtain an enriched population of full length mRNA molecules, which can further be used, for example, to produce cDNA molecules by contacting the mRNA molecules with a polypeptide having reverse transcriptase activity and, optionally, with a polypeptide having DNA polymerase activity. Accordingly, the present invention provides an enriched population of nucleic acid molecules prepared according to a method of the invention, for example, an enriched population of nucleic acid molecules having a 5′-cap, or an enriched population of full length mRNA molecules, or an enriched population of full length cDNA molecules, which can be single stranded or double stranded.

The present invention also relates to a method of obtaining an enriched population of mRNA molecules having a 5′-cap from a population of nucleotide sequences containing such RNA molecules. Such a method can be performed, for example, by contacting a sample containing a population of nucleotide sequences and at least one agent that selectively binds at or near a free 5′-phosphate group of a nucleotide sequence, under conditions that allow the agent to bind to a free 5′-phosphate group of a nucleotide sequence; and removing nucleotide sequences having the agent bound thereto from the sample, thereby obtaining an enriched population of RNA molecules comprising a 5′-cap. The agent preferably includes at least one first member of a specific binding pair, and the step of removing nucleotide sequences having the agent bound thereto includes contacting the sample with at least one second member of the specific binding pair, and removing nucleotide sequences in which the second member of the specific binding pair bound to the agent. The agent containing the first member of a specific binding pair preferably is a biotinylated oligoribonucleotide, and the second member of the specific binding pair includes avidin or streptavidin.

In one embodiment, removing nucleotide sequences having one or more second members of the specific binding pair bound to the agent involves contacting the sample with phenol/chloroform, wherein the first and second members of the specific binding pair, including agent and/or nucleotide sequences bound thereto partition into the organic phase, and removing the aqueous fraction, which contains RNA molecules comprising a 5′-cap. In another embodiment, the one or more second members of the specific binding pair (e.g., avidin or streptavidin) is coupled to a solid support, and removing nucleotide sequences having the second member of the specific binding pair bound to the agent involves contacting the sample with the solid support, and removing the sample, including unbound RNA molecules containing a 5′-cap, from the solid support.

The present invention further relates to a method of obtaining full length mRNA molecules, which contain a 5′-cap and a polyA tail, from a population of nucleotide sequences containing full length mRNA molecules. In one embodiment, the method is performed, for example, by contacting a sample containing the population of nucleotide sequences and at least one agent that selectively binds at or near a free 5′-phosphate group of a nucleotide sequence, under conditions that allow the agent to bind to a free 5′-phosphate group of a nucleotide sequence; removing nucleotide sequences having the agent bound thereto from the sample, thereby obtaining an enriched population of RNA molecules comprising a 5′-cap; contacting the enriched population of RNA molecules comprising a 5′-cap with at least one polynucleotide that selectively hybridizes to a 3′-polyadenosine nucleotide sequence, under conditions that allow selective hybridization; and isolating RNA molecules that selectively hybridize to the polynucleotide, thereby obtaining an enriched population of full length mRNA molecules comprising a 5′-cap and a polyA tail.

In another embodiment, the method of obtaining an enriched population of full length mRNA molecules is performed, for example, by contacting a sample containing the population of nucleotide sequences with at least one polynucleotide that selectively hybridizes to a 3′-polyadenosine nucleotide sequence, under conditions that allow selective hybridization; isolating nucleic acid molecules that selectively hybridize to the polynucleotide, thereby obtaining a population of nucleic acid molecules comprising a polyadenosine nucleotide sequence; contacting the population of nucleic acid molecules comprising a polyadenosine nucleotide sequence, and at least one agent that selectively binds at or near a free 5′-phosphate group of a nucleotide sequence, under conditions that allow the agent to bind to a free 5′-phosphate group of a nucleotide sequence; and removing nucleotide sequences having the agent bound thereto from the sample, thereby obtaining an enriched population of RNA molecules comprising a 5′-cap. Accordingly, the present invention also provides an enriched population of full length mRNA molecules comprising a 5′-cap and a polyA tail produced by either of such methods.

In accordance with the invention, an agent that selectively binds a free 5′-phosphate group of a nucleotide sequence can be used to remove such nucleotide sequences from a population that further contains 5′-capped nucleic acid molecules, thereby providing a means for obtaining an enriched population of 5′:capped nucleic acid molecules, particularly 5′-capped RNA molecules. The agent can facilitate removal of nucleotide sequences having a free 5′-phosphate by relying on the ability of a second molecule that can bind to the agent. In one embodiment, that agent comprises a first member of a specific binding pair, wherein a second member of the specific binding pair is used to facilitate removal of the agent, and any nucleotide sequences bound thereto, from a sample.

As disclosed herein, a full length mRNA molecule obtained according to a method of the invention can be used as a template for DNA synthesis by contacting the RNA template with one or more polypeptides having reverse transcriptase activity and a primer such as an oligodeoxythymidine (oligo dT) primer and incubating the mixture under conditions sufficient to make one or more cDNA molecules, which is complementary to all or a portion of one or more RNA template. Such conditions can include the use of one or more nucleotides, a suitable buffer, and one or more nucleic acid primers. The synthesized cDNA molecule can then be used as a template for second strand cDNA synthesis or for a DNA amplification reaction. Accordingly, a cDNA or cDNA library, particularly full length cDNA molecules, can be produced from the full length RNA molecules.

The present invention further relates to a kit, which contains one or more components useful for practicing a method of the invention. A kit of the invention can contain at least one agent that selectively binds to an uncapped nucleotide sequence, for example, an oligoribonucleotide agent, which can selectively bind at or near a 5′-phosphate group of an RNA molecule. If desired, the agent can comprise one or more members of a specific binding pair, or the kit can contain one or a variety of different members, which conveniently can be incorporated into the agent, in which case the kit also can contain one or more reagents for incorporating the member of the specific binding pair into the agent. The kit can comprise a carrier means such as a box, carton, or the like, which can be compartmentalized to receive in close confinement therein one or more containers, such as tubes, vials, bottles, ampules and the like, wherein a first container contains, for example, an agent such as an oligoribonucleotide, one or more ligand molecules such as biotin, and the like, and can further contain instructions for practicing one or more steps of a method of the invention. A kit of the invention also can contain reagents for practicing one or more steps of the invention, for example, an RNA ligase where the agent is an oligoribonucleotide, thereby providing a means to selectively bind the agent oligoribonucleotide to a 5′ phosphate group of an RNA molecule, or can contain a polypeptide having reverse transcriptase activity or other polymerase activity; or can contain one or more members of a specific binding pair, for example, second members of a specific binding pair where the agent comprises at least one first member of a specific binding pair, which can provide a means for removing an agent, and any RNA molecule bound thereto, from a sample. If desired, at least one second member of a specific binding pair, which can be included in the kit, can be coupled to a solid support. As such, the kit can contain solid supports having one or more second members of a specific binding pair coupled thereto, or can contain one or a variety of solid supports and one or a variety of second members of a specific binding pair, and, if desired, reagents for coupling the member to a solid support.

### BRIEF DESCRIPTION OF THE DRAWINGS

FIG. 1 illustrates a general experimental method for comparing the disclosed method of producing a full length cDNA library with the standard (“regular”) method.

FIG. 2 sets forth a procedure for isolating intact, full length mRNA molecules using a method of the invention.

FIG. 3 shows the distribution of 5′ end positions for clones obtained using the method of the invention (“full length cDNA library”) and the standard method (“regular cDNA library”).

FIG. 4 provides a comparison of the length of mRNA molecules obtained using a method of the invention (“negative selection”) and the standard method (“regular procedure”).

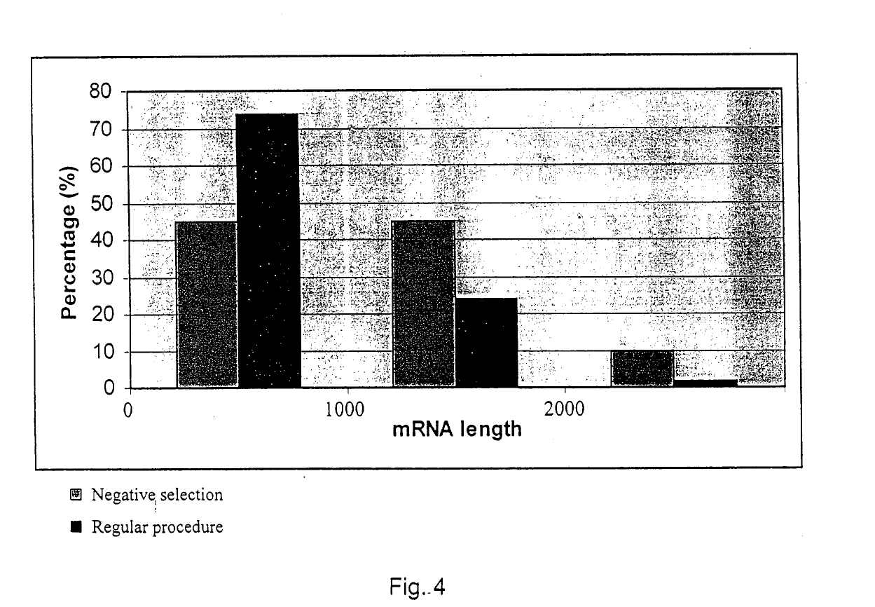

### DETAILED DESCRIPTION OF THE INVENTION

The present invention provides methods for obtaining enriched populations of nucleic acid molecules having a 5′-cap from a population of nucleotide sequences, including nucleic acid molecules comprising a 5′-cap. As disclosed herein, the methods are particularly useful for obtaining full length mRNA molecules, which contain a 5′-cap and a polyA tail. Also disclosed herein are uses for such mRNA molecules, including for preparing full length cDNA molecules and libraries of such cDNA molecules.

Construction of full length cDNA libraries is an essential step for the study of gene function. The method for selecting the intact mRNA directly affects the number of full length transcripts, and many methods have been developed for obtaining intact mRNA molecules containing a 5′-cap structure. Full length cDNA library construction is continually evolving and is becoming more critical as the field of gene functional analysis progresses. Many different methods have been developed, most being focused on the 5′-cap modification that is common to mRNA molecules of eukaryotic organisms. Such methods generally have been “positive selection” methods, that target the 5′-cap structure. The methods as disclosed herein are distinguishable in that a “negative selection” method is provided, wherein nucleic acid molecules containing a 5′-cap are obtained in an enriched form by targeting and removing nucleic acid molecules are uncapped and, more preferably, by targeting and removing nucleic molecules that have a free 5′-phosphate group and, therefore, lack a 5′-cap.

As disclosed herein, the negative selection method of the invention substantially removes both uncapped mRNA and other non-mRNA nucleic acid molecules that contain a free 5′-phosphate. Briefly, a standard mRNA purification procedure using an oligo dT column extraction was performed, then a biotinylated oligoribonucleotide was ligated to free 5′-phosphate groups of nucleotide sequences using T4 RNA ligase. Streptavidin extraction and phenol/chloroform purification were performed, and all truncated mRNA and other nucleic acid molecules were removed from the intact full length mRNA. This methodology has been applied to the construction of a mouse brain full length cDNA library. When random sequencing results of the sequences from a mouse brain cDNA library generated by standard mRNA purification procedure were compared to the cDNA library prepared using the disclosed method, there was a significant increase in the representation of full length clones obtained using the method of the invention. The sequence analysis of 302 clones from the full length library revealed that 41% of the clones were known genes, and within these known genes, the full length clones totaled more than 68% with the 5′ end positions ranging between −485 to +100. In addition, the full length cDNA library showed a novel gene discovery rate of 11% when compared to GenBank. The largest mRNA size confirmed by sequencing was 4 kilobases (kb). In comparison, the cDNA library prepared using the standard method had 130 sequences confirmed, and included 3% novel genes and 74% known genes. Within the 96 known genes, there were 31 full length clones (33%) with 5′ end positions ranging between −233 to +100. The largest mRNA size was 2.9 kb. The statistical analysis showed that there were significant differences between two libraries in both 5′ end position and mRNA size (p<0.05).

Accordingly, the present invention provides methods of obtaining 5′-capped nucleic acid molecules, including full length mRNA molecules. A method of the invention can be performed, for example, by contacting a sample containing a population of nucleotide sequences and at least one agent that selectively binds at or near a free 5′-phosphate group of a nucleotide sequence, under conditions that allow the agent to selectively bind at or near a free 5′-phosphate group of a nucleotide sequence, and removing the agent and any nucleotide sequence bound thereto from the sample. The agent can be any molecule that selectively binds at or near a 5′-phosphate group of a nucleotide sequence, including, for example, an oligonucleotide, a peptide, or a small organic molecule. The agent can bind directly to the nucleotide sequence, or binding can be mediated through a chemical or enzymatic reaction. For example, that the agent can be an oligonucleotide, and selective binding of the oligonucleotide to a 5′-phosphate group of a nucleotide sequence can be mediated by a ligase.

In one aspect, a method of the invention utilizes an oligoribonucleotide as an agent that can selectively bind nucleic acid molecules, including RNA molecules, having a free 5′-phosphate group. Selective binding of the oligoribonucleotide agent can be effected by contacting the agent, which contains a free reactive hydroxyl group, a population of nucleotide sequences, which include nucleotide sequences containing a free 5′-phosphate group, such that the RNA ligase can link the agent oligoribonucleotide to nucleotide sequences containing a free 5′-phosphate group. The molecule having RNA ligase activity can be, for example, a polypeptide such as T4 RNA ligase, which can link an oligoribonucleotide containing a 3′-hydroxyl group to a nucleotide sequences having a free 5′-phosphate, or E. coli 2′-5′ RNA ligase, which can link an oligoribonucleotide containing a 2′-hydroxyl group to a nucleotide sequences having a free 5′-phosphate (Arn and Abelson, J. Biol. Chem. 271:31145-31153, 1996, which is incorporated herein by reference); or a ribozyme that has RNA ligase activity (Wilson and Szostak, Ann. Rev. Biochem. 68:611-647, 1999, which is incorporated herein by reference).

A method of the invention requires a means for removing nucleotide sequences having an agent bound thereto from a sample, such that an enriched population of nucleic molecules containing a 5′-cap can be obtained. The removing step can be performed using at least one moiety that selectively binds the agent. For example, where the agent is an oligonucleotide, the moiety can be a polynucleotide having sufficient complementarity that it can selectively hybridize to the oligonucleotide agent, thereby providing a means to remove the agent, and any nucleotide sequences bound thereto, from the sample. Where the agent is a peptide or polypeptide, or other antigenic molecule, the moiety can be an antibody that specifically binds the peptide agent. If desired, one or more moieties can be coupled to a solid support, which can facilitate removal of the agent and any nucleotide sequences bound thereto from the sample. As such, it should be recognized that, while reference is made herein to “removal” of uncapped nucleotide sequences or of nucleotide sequences containing a free 5′-phosphate group, the method can be performed by removing either the uncapped nucleotide sequences or the 5′-capped nucleic acid molecules from the sample. For example, where one or more second members of a specific binding pair are coupled to a vessel containing the sample (e.g., a well of a microtiter plate), the sample including 5′-capped nucleic acid molecules can be removed from the vessel using a pipet, thus leaving behind uncapped nucleotide sequences having agent comprising at least one first member of the specific binding pair bound thereto. Such a procedure is nevertheless considered to be removal of uncapped nucleotide sequences.

Preferably, the agent comprises at least one first member of a specific binding pair, wherein at least one second member of the specific binding pair is used to facilitate removal of the agent, and any nucleotide sequences bound thereto, from the sample. As used herein, the term “specific binding pair” refers to two (or more) molecules that can specifically interact with each other. The two (or more) molecules of a specific binding pair are referred to as “members of a specific binding pair” or as “binding partners.” A specific binding pair is selected such that the interaction is stable under conditions generally used to perform a method of the invention. Although reference is made herein to a “first” and a “second” member of a specific binding pair, such terms are used only for convenience and clarity of discussion, and it will be recognized that generally either the “first” or “second” (or other) member of a specific binding pair can be incorporated into an agent and the other member can be coupled to a solid support.

Specific binding pairs are well known in the art and include, for example, an antibody that specifically interacts with an epitope and the epitope, for example, an anti-FLAG antibody and a FLAG peptide (Hopp et al., BioTechnology 6:1204 (1988); U.S. Pat. No. 5,011,912); glutathione and glutathione S-transferase (GST); a divalent metal ion such as nickel ion or cobalt ion and a polyhistidine peptide; biotin and avidin or streptavidin, and the like. Additional examples of specific binding pairs that can be used according to a method of the invention include an enzyme and its substrate; a lipopolysaccharide and specific receptor; apbtransferrin or ferrotransferrin and iron ion; insulin and an insulin receptor; cytokines, growth factors, and the like, and a specific receptor for the polypeptide; gp120; a molecule such as laminin, collagen, fibronectin, vitronectin, or an integrin such as αᵥβ₁, αᵥβ₃, α₃β₁, α₄β₁, α₄β₇, α₅β₁, αᵥβ₁, α_{IIb}β₃, αᵥβ₃; αᵥβ₆, α₁β₁, α₂β₁, α₃β₁, αᵥβ₃, α₁β₁, α₂β₁, α₃β₁, α₆β₁, α₇β₁ or α₆β₅ and a ligand containing an RGD tripeptide; protein A, protein G, or a cell-surface Fc receptor and an antibody; and the like.

Biotin and streptavidin or biotin and avidin are examples of specific binding pairs that can be particularly useful in the methods of the invention, in that they provide the advantage that a single avidin or streptavidin molecule can bind four biotin moieties, and that the binding occurs with a very high affinity, thus facilitating removal of nucleotide sequence having an agent comprising biotin bound thereto. Furthermore, biotin can be conveniently incorporated into various molecules that can be useful as an agent in a method of the invention. For example, biotinylated nucleotides are available and can readily be incorporated into an oligonucleotide, and biotin can readily be incorporated into a peptide by chemically linking it to a lysine residue or through an enzymatic reaction, wherein the peptide comprises a signal sequence comprising a biotinylation site for the enzyme BirA.

A first member of the specific binding pair generally is incorporated into the agent, and the second member of the specific binding pair generally is coupled to a solid support. A member of a specific binding pair can be incorporated into an agent either by including the member as a component of the reagents used to synthesize the reagent, for example, using biotinylated-uridine in a method of chemically synthesizing a biotinylated oligoribonucleotide, or by chemically or enzymatically coupling the member of the specific binding pair to the agent. Methods for performing such a chemical or enzymatic coupling will be selected based on the chemical or biological properties of the agent and the member of the specific binding pair, and generally will use conventional methods of organic or biological synthesis. For example, in addition to incorporating biotin into an oligonucleotide during synthesis or enzymatically, as described above, an oligonucleotide also can be biotinylated at the 5′ terminus by first producing 5′ amino (NH2) groups followed by Cab-NHS ester addition (Langer et al., Proc. Natl. Acad. Sci., USA 78:6633, 1981).

Any solid support to which a member of a specific binding pair can be coupled and, thereby immobilized, can be used in practicing the methods of the invention. For example, useful solid supports include, but are not limited to, nitrocellulose, diazocellulose, glass, polystyrene, polyvinylchloride, polypropylene, polyethylene, dextran, SEPHAROSE™ gel, agar, starch, and nylon, and can be in any of various forms, including, for example, as beads, slides, or wells of a microtiter plate. Preferred solid supports include beads made of glass, latex or a magnetic, paramagnetic or superparamagnetic material. Coupling of the member of the specific binding pair to the solid support can be accomplished by any method, including, for example, a covalent, hydrophobic, or ionic coupling, including coating. For example, where an agent comprises biotin, the solid support, which contains avidin or streptavidin coupled thereto, can be magnetic, paramagnetic or superparamagnetic beads (Dynal A. S.; Oslo Norway; Sigma; St. Louis Mo.).

To remove nucleotide sequences containing a free 5′-phosphate, the sample containing the nucleotide sequences is contacted with the agent, which can comprise a member of a specific binding pair such as biotin, under conditions such that the agent can selectively bind nucleotide sequences having a free 5′-phosphate group, then the sample is further contacted with the second member of the specific binding pair, which generally is coupled to a solid support. Typically, the conditions under which the reactions are performed include incubation in a buffered solution such as a TRIS, phosphate, HEPES or carbonate buffered solution at about a pH of 6 to 9, preferably about pH of about 7 to 8, and also can contain sodium chloride, EDTA, a reducing agent such as β-mercaptoethanol, a metal ion cofactor, or the like. Incubation is performed at about 0° C. to about 37° C., for about 30 minutes to overnight, depending on the particular step. For example, where an agent is an oligonucleotide that is ligated to free 5′-phosphate, the reaction generally is performed overnight at room temperature. In comparison, a reaction involving the contact of an agent comprising biotin to a solid support comprising streptavidin proceeds rapidly and, therefore, can be completed after about 30 minutes to 1 hour.

Upon contacting the agent, and any bound nucleotide sequences, to the solid support, the unwanted bound nucleotide sequences can be removed from the desired 5′-capped nucleic acid molecules either by removing the solid support from the sample or by removing the sample from the solid support. For example, wherein the agent comprises a biotinylated oligonucleotide that contains a free 3′ hydroxyl group and is ligated to a free 5′-phosphate group of a nucleotide sequence, avidin or streptavidin can be coupled to a solid support such as agarose beads, which can be contained in a chromatography column, then the sample can be passed over the column and the flow through fraction containing 5′-capped nucleic acid molecules can be collected. Alternatively, the avidin or streptavidin can be coupled to magnetic beads, which can be placed into the sample, then either removed using a magnet (for example, a Magna-Sep Magnetic Particle Separator; Invitrogen Corp.; Carlsbad Calif.), thereby leaving in the sample an enriched population of 5′-capped nucleic acid molecules, or the magnet can be used to maintain the beads in the vessel and the sample, which is enriched for 5′-capped nucleic acid molecules can be withdrawn using a pipette. If desired, the solid support subsequently can be washed one or more times to remove any non-specifically bound 5′-capped nucleic acid molecules.

An enriched population of 5′-capped nucleic acid molecules can be obtained according to a method of the invention beginning with any population of nucleotide sequences that include nucleic acid molecules, particularly RNA molecules comprising a 5′ cap. As such, the population of nucleotide sequences can be obtained from cells, tissues or organs of any organism containing 5′-capped RNA, particularly eukaryotic organisms such as fungi, including yeast; plants; protozoans and other eukaryotic parasitic organisms; invertebrate organisms such as insects (e.g., Drosophila spp.) and nematodes (e.g. Caenorhabditis elegans), and vertebrates, including fish, birds, reptiles, and mammals, particularly cells, tissues or organs from a human. Generally, though not necessarily, genomic DNA is removed from an initially obtained population of nucleotide sequences using routine methods. Preferably, the population of nucleotide sequences is a population of RNA sequences, which can include transfer RNA, ribosomal RNA, and mRNA.

Somatic cells, including mammalian somatic cells, that can be used as a source of a population of nucleotide sequences include blood cells (reticulocytes and leukocytes), endothelial cells, epithelial cells, neuronal cells (from the central or peripheral nervous system), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells, for example, macrophages, dendritic cells, Schwann cells. Mammalian germ cells (spermatocytes and oocytes) also can be used as a source of nucleotide sequences for use in a method of the invention, as can the progenitor, precursor and stem cells that give rise to the such somatic and germ cells. Also suitable for use are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

The cells, tissues or organs can be obtained from a normal (healthy) or diseased individual, including, for example, transformed cells, or tumor cells, which can be benign tumor cells or malignant tumor cells. Diseased cells may, for example, include those involved in infectious diseases (caused by bacteria, fungi or yeast, viruses such as HIV, or parasites), in genetic or biochemical pathologies such as cystic fibrosis, hemophilia, Alzheimer's disease, muscular dystrophy, multiple sclerosis, or a cancer. Transformed or established animal cell lines include, for example, COS cells, CHO cells, VERO cells, BHK cells, HeLa cells, HepG2 cells, K562 cells, F9 cells and the like. Once the starting cells, tissues, organs or other samples are obtained, the nucleotide sequences, including DNA and RNA, can be isolated therefrom using routine methods (see, for example, Sambrook et al., “Molecular Cloning: A laboratory manual” (Cold Spring Harbor Laboratory Press 1988)).

As disclosed herein, the population of nucleotide sequences generally is then contacted with the agent, which provides a means to remove RNA or other nucleotide sequences that have a free 5′-phosphate group and, therefore, lack a 5′-cap, from those nucleic acid molecules, particularly RNA molecules, that contain a 5′-cap. However, one or more steps can be performed on the population of nucleotide sequences including, for example, a step of isolating RNA molecules containing a polyadenosine nucleotide sequence, particularly a polyA tail.

An enriched population of 5′-capped RNA molecules, for example, then can be used in a 5′ RACE procedure to obtain information about the 5′ end of the capped RNA molecules, or can be used to prepare cDNA, in which case, if desired, the enriched population of 5′-capped RNA molecules can be further selected to obtain those 5′-capped RNA molecules that also contain a polyA tail, thus providing a means to obtain full length mRNA molecules, which comprise a 5′-cap and a polyA tail. Using the methods of the invention, enriched populations of 5′-capped nucleic acid molecules, particularly 5′-capped RNA molecules, and more particularly, full length mRNA molecules, which contain a 5′-cap and a polyA tail, such nucleic acid molecules being useful for preparing full length cDNA molecules.

For cDNA synthesis, first and, if desired, second strand cDNA reactions generally, though not necessarily, can be performed in one tube, then can be isolated using any conventional method. The cDNA then can be further manipulated, including by cloning into a vector. Subsequent or prior to cloning into a vector, specific cDNA sequences can be isolated using a specific oligonucleotide and standard hybridization methods. As such, full length cDNA molecules or libraries of cDNA molecules can be obtained. Accordingly, the present invention provides full length cDNA molecules and libraries of such cDNA molecule produced according to such a method.

A cDNA molecule or library of cDNA molecules can be produced by mixing full length mRNA molecules obtained as disclosed herein with one or more polypeptides having polymerase activity and/or reverse transcriptase activity and with a one or more primers. Under conditions favoring the reverse transcription and/or polymerization of the input full length RNA molecule, synthesis of a nucleic acid molecule complementary to all or a portion of the template is accomplished. Polypeptides, which are enzymes having reverse transcriptase and/or polymerase activity, useful for preparing a cDNA from an RNA template or a second cDNA strand from a first cDNA strand template include, but are not limited to, Moloney Murine Leukemia Virus (M-MLV) reverse transcriptase, Rous Sarcoma Virus (RSV) reverse transcriptase, avian myeloblastosis virus (AMV) reverse transcriptase, Rous associated virus (RAV) reverse transcriptase, myeloblastosis associated virus (MAV) reverse transcriptase, human immunodeficiency virus (HIV) reverse transcriptase, retroviral reverse transcriptase, retrotransposon reverse transcriptase, hepatitis B reverse transcriptase, cauliflower mosaic virus reverse transcriptase, bacterial reverse transcriptase, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™ polymerase) DNA polymerase, Pyrococcus furiosus (Pfu or DEEPVENT™ polymerase) DNA polymerase, Pyrococcus woosii (Pwo) DNA polymerase, Bacillus sterothermophilus (Bst) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™ polymerase) DNA polymerase, Methanobacterium thermoautotrophicum (Mth) DNA polymerase, and mutants, variants and derivatives thereof. Particularly useful are variants of these enzymes that have substantially reduced RNase H activity. By an enzyme “substantially reduced in RNase H activity” is meant that the enzyme has less than about 20%, particularly less than about 15%, 10% or 5%, and preferably less than about 2%, of the RNase H activity of the RNAse H activity of a corresponding naturally occurring enzyme such as a naturally occurring M-MLV or AMV reverse transcriptase. The RNase H activity of any enzyme can be determined by a variety of assays (see, for example, U.S. Pat. No. 5,244,797; Kotewicz et al., Nucl. Acids Res. 16:265, 1988; Gerard et al., FOCUS 14(5):91, 1992, each of which is incorporated herein by reference).

Nucleic acid molecules additionally can be produced from the enriched population of 5′-capped nucleic acid molecules, including, if desired, the full length mRNA molecules, using other methods such as a primer extension reaction. Such a method involves hybridization of a primer to the 5′-capped nucleic acid molecule templates, and extending the primer to generate a nucleic acid molecule complementary to all or a portion of the template. Such synthesis is accomplished in the presence of nucleotides, including deoxyribonucleoside triphosphates (dNTPs) and, if desired, dideoxyribonucleoside triphosphates (ddNTPs) or derivatives thereof), and one or more polypeptides having polymerase and/or reverse transcriptase activity.

As disclosed herein, the “negative selection” method substantially removes from a population of nucleotide sequences, those sequences containing free 5′-phosphate group, thereby providing an enriched population of 5′-capped nucleic acid molecules, particularly 5′-capped RNA molecules, including intact, full length mRNA molecules containing a 5′-cap and a polyA tail. The 5′-cap structure generally stabilizes mRNA by protecting it against 5′-exonucleolytic degradation (Furuichi et al., Nature 266:235-239,1977; Shimotohno et al., Proc. Natl. Acad. Sci., USA 74:2734-8, 1977). Thus, by obtaining populations of RNA molecules having a 5′-cap, the likelihood of full length mRNA molecules being present is increased. As disclosed herein, the preparation of two cDNA libraries was performed in parallel, wherein all the steps were identical except that one cDNA library was prepared using mRNA obtained using the negative selection method of the invention (“full length library”) and the other cDNA library was prepared using mRNA obtained by the standard method currently used in the art (“regular library”).

A total of 432 random clones, including 302 from full length library and 130 from the regular library, were sequenced from the 5′ end using M13 reverse primer (see Example 1). To estimate the ratio of the clones with internal first strand priming or internal cloning sites, 150 clones from the full length cDNA library were also sequenced from the 3′ end using M13 forward primer (Example 1) to determine the percentage of cDNA clones that did not have poly A tails. Only 2% of the cDNA clones fit this profile. The sequencing result analysis was concentrated in two fields, 5′-end position and mRNA length. The start point of the coding region was defined as +1, and every site beyond (3′ or downstream of) the start point as a positive position. The limit for the positive position was set at +200 for those clones that were closing to the start point. The negative position represented a full length clone. The results indicated that the major distribution of the 5′ end positions were from −150 to +50 in the full length cDNA library and from −50 to +150 in the regular cDNA library. The mRNA length was determined according to those in the GenBank database.

In addition, two-thirds of the clones from the regular cDNA library had a length less than 1000 bp, and only 2% of the mRNAs from the regular cDNA library were larger than 2000 bp, whereas 45% of the clones from the full length cDNA library had a length between 1000 to 2000 bp and 10% were larger than 2000 bp. The longest mRNA from the sequenced clones was 3957 bp in the full length cDNA library and 2904 bp in the regular cDNA library. Comparing the random clone sequencing results of the two cDNA libraries, the representative ratio of real full length clones in the full length cDNA library was approximately three times more than that in the regular cDNA library. Statistical analysis of a total of 219 known genes from both libraries indicated that there were significant differences between the two libraries in both the distributions of the 5′ end position and mRNA length. These results demonstrate that the “negative selection” procedure increased the representative ratio of intact mRNA molecules and, therefore, full length cDNA clones. Accordingly, the “negative selection” procedure generally provides a simpler and more cost effective method than most positive selection techniques, as there are no requirements for specific reagents and equipment, and all the reagents and enzymes are commercially available and affordable.

The present invention also provides kits for use in practicing a method of the invention. Kits according to this aspect of the invention comprise a carrier means, such as a box, carton, tube or the like, having in close confinement therein one or more containers, such as vials, tubes, ampules, bottles and the like, wherein a first container contains one or more agents that selectively bind an uncapped nucleic acid molecule, preferably a nucleotide sequence having a free 5′-phosphate group, wherein the one or more agents can, but need not comprise, one or more first members of a specific binding pair. In other aspects, the kits of the invention may further comprise one or more additional containers containing a solid support, to which a one or more members of one or more specific binding pairs can be coupled. In additional aspects, the kits of the invention can further comprise one or more additional containers containing, for example, one or more nucleotides (e.g., dNTPs, ddNTPs or derivatives thereof) or one or more polypeptides (e.g., enzymes) having reverse transcriptase activity and/or polymerase activity, preferably any of those enzymes described above. Such nucleotides or derivatives thereof may include, but are not limited to, dUTP, dATP, dTTP, dCTP, dGTP, dITP, 7-deaza-dGTP, α-thio-dATP, α-thio-dTTP, α-thio-dGTP, α-thio-dCTP, ddUTP, ddATP, ddTTP, ddCTP, ddGTP, ddITP, 7-deaza-ddGTP, α-thio-ddATP, α-thio-ddTTP, α-thio-ddGTP, α-thio-ddCTP or derivatives thereof, all of which are available commercially from sources including Life Technologies, Inc. (Rockville, Md.), New England BioLabs (Beverly, Mass.) and Sigma Chemical Company (Saint Louis, Mo.). The kits encompassed by this aspect of the present invention may further comprise additional reagents (e.g., suitable buffers) and compounds necessary for carrying out nucleic acid reverse transcription and/or polymerization protocols.

The present invention can be used in a variety of applications requiring 5′-capped nucleic acid molecules, particularly 5′-capped mRNA, including 5′-capped mRNA molecules containing a polyA tail. The invention is also directed to methods for the amplification of a nucleic acid molecule, and to nucleic acid molecules amplified by to these methods. According to this aspect of the invention, a nucleic acid molecule may be amplified (i.e., additional copies of the nucleic acid molecule prepared) by amplifying the nucleic acid molecule (e.g., a cDNA molecules) of the invention according to any amplification method that is known in the art. Particularly preferred amplification methods according to this aspect of the invention include PCR (U.S. Pat. Nos. 4,683,195 and 4,683,202), strand displacement amplification (SDA; U.S. Pat. No. 5,455,166; EP 0 684 315), and nucleic acid sequence-based amplification (NASBA; U.S. Pat. No. 5,409,818; EP 0 329 822). Most preferred are those methods comprising one or more PCR amplifications.

The invention is also directed to methods that may be used to prepare recombinant vectors which comprise the nucleic acid molecules or amplified nucleic acid molecules prepared according to a method of the invention, to host cells which comprise these recombinant vectors, to methods for the production of a recombinant polypeptide using these vectors and host cells, and to recombinant polypeptides produced using these methods.

Recombinant vectors may be produced according to this aspect of the invention by inserting, using methods that are well-known in the art, one or more of the nucleic acid molecules or amplified nucleic acid molecules prepared according to the present methods into a vector. The vector used in this aspect of the invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host. Preferably, the vectors are expression vectors that provide for specific expression of the cDNA molecule or nucleic acid molecule of the invention, which vectors may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids or bacteriophages, and vectors derived from combinations thereof, such as cosmids and phagemids, and will preferably include at least one selectable marker such as a tetracycline or ampicillin resistance gene for culturing in a bacterial host cell. Prior to insertion into such an expression vector, the nucleic acid molecules (e.g., cDNA molecules) or amplified nucleic acid molecules of the invention should be operatively linked to an appropriate promoter, such as the phage lambda PL promoter, the E. Coli lac, trp and tac promoters. Other suitable promoters will be known to the skilled artisan. Among vectors preferred for use in the present invention include pQE70, pQE60 and pQE-9 vectors, available from Qiagen; pBS vectors, PHAGESCRIPT™ vectors, BLUESCRIPT™ vectors, pNH8A, pNH16a, pNH18A, pNH46A vectors, available from Stratagene; pcDNA3 available from Invitrogen; pGEX, pTrxfus, pTrc99a, pET-5, pET-9, pKK223-3, pKK233-3, pDR540, pRIT5 vectors available from Pharmacia; and pSPORT1, pSPORT2 and pSV.SPORT1 vectors, available from Life Technologies, Inc. Other suitable vectors will be readily apparent to the skilled artisan.

Representative host cells that may be used according to the invention include, but are not limited to, bacterial cells, yeast cells, plant cells and animal cells. Preferred bacterial host cells include Escherichia spp. cells (particularly E. coli cells and most particularly E. coli strains DH10B and Stb12), Bacillus spp. cells (particularly B. subtilis and B. megaterium cells), Streptomyces spp. cells, Erwinia spp. cells, Klebsiella spp. cells and Salmonella spp. cells (particularly S. typhimurium cells). Preferred animal host cells include insect cells (most particularly Spodoptera frugiperda Sf9 and Sf21 cells and Trichoplusa High-Five cells) and mammalian cells (most particularly CHO, COS, VERO, BHK and human cells). These and other suitable host cells are available commercially, for example from Invitrogen Corp., American Type Culture Collection, and the like.

In addition, the invention provides methods for producing a recombinant polypeptide, and polypeptides produced by these methods. According to this aspect of the invention, a recombinant polypeptide may be produced by culturing any of the above recombinant host cells under conditions favoring production of a polypeptide therefrom, and isolation of the polypeptide. Methods for culturing recombinant host cells, and for production and isolation of polypeptides therefrom, are well-known to one of ordinary skill in the art. In other applications, the methods of the invention may be used to generate a gene-specific cDNA library from a complex population of poly A+ RNA. In particular, the methods of the invention are particularly useful when the mRNA of interest represents only a minute fraction of the total RNA because the mRNA obtained can be efficiently reverse transcribed to produce full length cDNA molecules.

Each of the references cited herein is expressly incorporated herein by reference. The following example is intended to illustrate but not limit the invention.

### EXAMPLE I

### Negative Selection of Full Length mRNA

This example demonstrates that the negative selection method provides a means to obtain enriched populations of full length mRNA molecules.

A. Materials and Methods

Total RNA Isolation

Total cellular RNA was isolated from mouse C57BL/6J brain (male) and human normal ileum (female, 61 years old) tissues by a modification of a standard procedure (Cbomczynski et al., Anal Biochem. 162:156-159, 1987; Puissant et al., BioTechniques 8:148-9, 1990). One gram of tissue was homogenized in 5 ml of Solution D (4 M guanidinium thiocyanate, 25 mM sodium citrate (pH 7.0), 0.5% sarkosyl, 0.1 M 2-mercaptoethanol) utilizing a Polytron PT3000 Homogenizer (BRINKMANN), then 500 μl of 2 M sodium acetate (pH 4.0) was added to the homogenate. After a phenol/chloroform (5/1) extraction, 2.5 ml each of isopropanol and 1.2 M sodium chloride/0.8 M sodium citrate were added to the aqueous phase.

The sample was centrifuged at 10,000 rpm for 15 min at 4° C. (Sorvall SS-34 rotor). The pellet was resuspended in 5 ml of Solution D and precipitated in 5 ml of isopropanol at −20° C. for 1 hr. The RNA pellet was isolated by centrifugation at 10,000 rpm for 20 min at 4° C., then washed with 70% ethanol. The pellet was resuspended in 2 ml of 10 mM Tris (pH 7.5)/1 mM EDTA/0.5% SDS and extracted with 2 ml of chloroform. The purified RNA was precipitated by adding 0.1 volumes of 3 M sodium acetate (pH 5.2) and 1 volume of isopropanol. After a 1 hr incubation at −20° C., the RNA pellet was collected by centrifugation at 10,000 rpm for 30 min at 4° C., then washed with 70% ethanol. The RNA was resuspended in DEPC treated water. Electrophoresis and spectrophotometry (O.D.260/280) were used to determine RNA quality and quantity.

Preparation of Full Length mRNA

300-500 μg of total RNA was applied an Oligo-(dT)-cellulose column (New England Biolabs) and polyA+ mRNA was isolated using the standard procedure (Aviv et al., Proc. Natl. Acad. Sci., USA 69:1408-12, 1972). Two μg of purified poly (A)⁺ mRNA was then ligated with 6 μg of biotinylated oligo-ribonucleotide (5′-GAGACGAUUUACGAUUUACG-3′; SEQ ID NO:1; U was biotinylated) using T4 RNA ligase (Promega). The ligation was performed at 20° C. for more than 16 hr in the presence of 200 units of RNase inhibitor (Roche). The ligation was stopped by the addition of proteinase K and incubation at 50° C. for 20 min. 260 μg of streptavidin was applied to the reaction mixture at room temperature and incubation was continued for 30 min. Following a phenol/chloroform extraction, purified full length mRNA was precipitated by the addition of 3 M sodium acetate (pH 5.2) and 100% ethanol.

Construction of cDNA Library

A regular cDNA library and a full length library were constructed from mouse brain mRNA as described (Soares, Proc. Natl. Acad. Sci., USA 91:9228-32, 1994). One μg of purified full length mRNA was denatured at 65° C. for 10 min. A reverse transcription reaction was initiated using an oligo dT primer containing a built-in Not I restriction site. The reaction was carried out using M-MLV Reverse Transcriptase and RNase H minus (Promega) for 2 hr. Subsequently, RNase H, E. coli DNA polymerase I, and E. coli DNA ligase were added for second strand cDNA synthesis. Double stranded cDNAs were made blunt ended, and linked with an Eco RI adapter (Amersham-Pharmacia) after size selection. Following Not I restriction digestion, the cDNAs were cloned into pT7T3D-Pac vector (phagemid).

DNA Sequencing

Plasmid DNA was isolated using an Autogen 740 Automatic Plasmid Isolation System (Integrated Separation Systems). DNA sequences were determined by the dideoxy termination method (Sanger et al., Proc. Natl. Acad. Sci., USA 74:5463-5467, 1977). Sequencing from the 5′ end used the M13 reverse primer (AACAGCTATGACCATG; SEQ ID NO:2), and sequencing from the 3′ end used the M13 forward primer (GTAAAACGACGGCCAGT; SEQ ID NO:3). The DNA sequences were read by an ABI Prism 3700 DNA Analyzer (PE Applied Biosystems).

B. Results

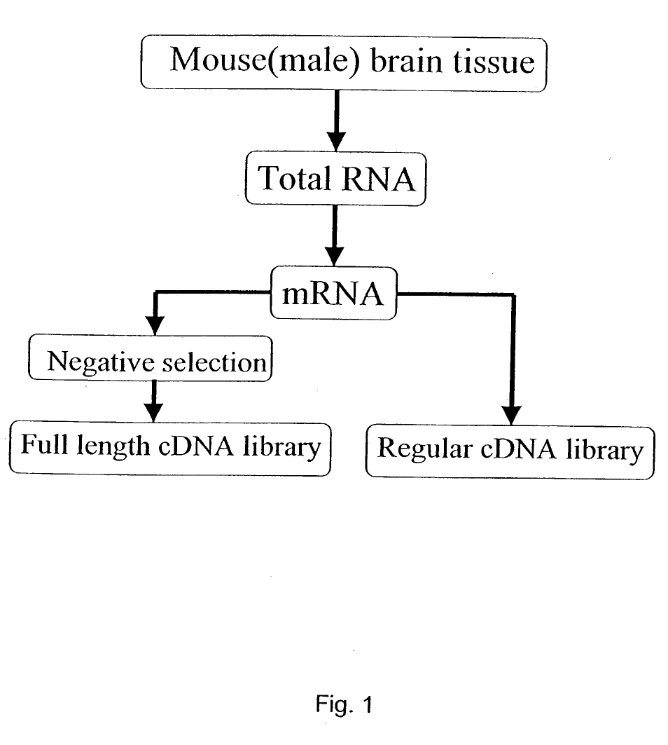

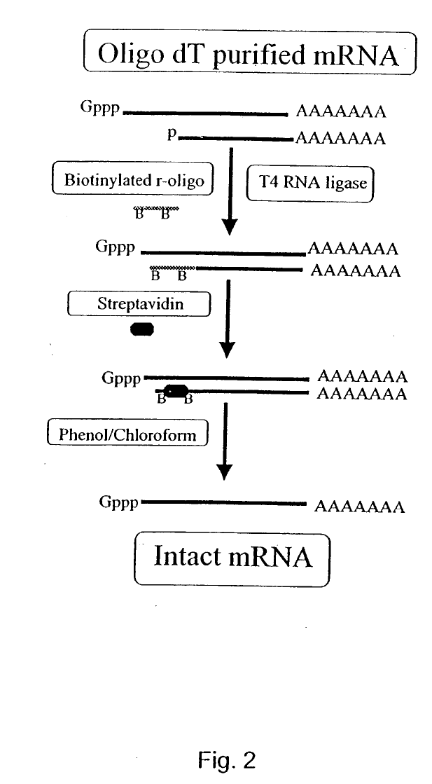

The experimental strategy is shown in FIG. 1 and a detailed procedure for negative selection of intact, full length mRNA is shown in FIG. 2. The full length library construction required only three additional steps as compared with the procedures of regular library construction, prior to the initiation of first strand synthesis. The average insert size of each library was determined by autoradiography immediately after size selection. An average size of about 1,500 bp was detected for the regular library and of about 2,000 bp for the full length library. The recombinant clone numbers before first round amplification were about 1.6×10⁶ cfu (colony forming units) for the regular library and about 350,000 cfu for the full length library.

After first round amplification, cDNA clones were randomly selected from both libraries for sequencing analysis. A total of 432 clones were sequenced from the 5′ end. Within them, 150 clones were sequenced from both ends; 2% had lost the poly T tails. All sequences were submitted to the GenBank for BLAST search. 41% of the clones of the full length library matched known genes and 11% of the clones were novel genes.

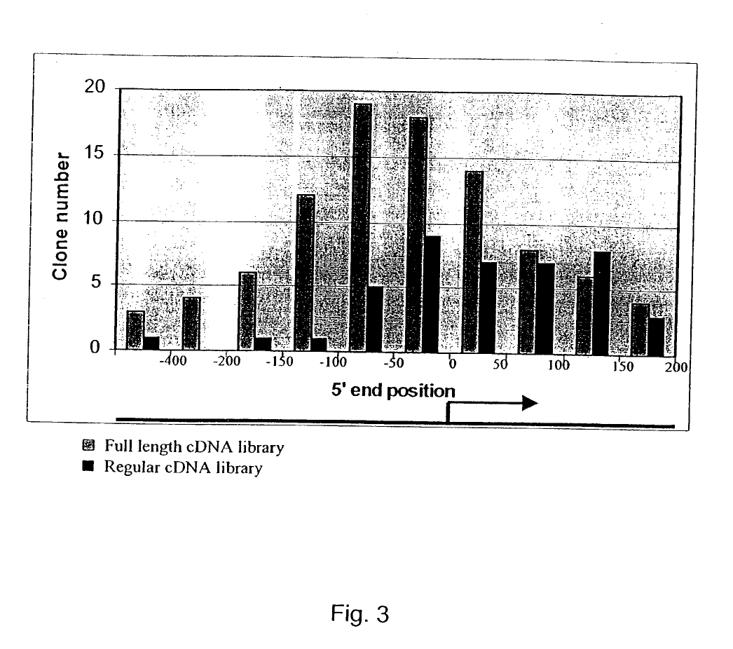

The analysis result of 5′ end distribution for each library is shown in FIG. 3. The start point of the coding sequence was set as the origin (arrow); positive numbers represent sequence that is beyond the start point. In the full length library, 50% of clones were full length (<0) and 68% of clones were not beyond the +100 position. For the regular library, only 18% of the clones were full length and 33% of the clones were within the +100 position (Table 1).

In order to determine if the differences between the two libraries were significant, a T-test for two-sample assuming equal variances was applied in the statistical study of the data for 5′ end position and mRNA size groups. The alpha value was set at 0.05. The t values were −2.17625 for the 5′ end position group and 3.265533 for the mRNA length group. Both groups had p values less than 0.05. These results indicated that the original hypothesis was not correct, i.e., the data in both groups had significant differences.

These results demonstrate that the negative selection methods results in the isolation of significantly more full length mRNA molecules that the standard mRNA isolation method.

TABLE I
Known gene distribution

| 5′ end | Full length | Regular |
|---|---|---|
| position | cDNA library | cDNA library |
| <0 | 62 (50%) | 17 (18%) |
| <50 | 76 (61%) | 24 (25%) |
| <100 | 84 (68%) | 31 (33%) |
| <150 | 90 (73%) | 39 (41%) |
| <200 | 94 (76%) | 42 (44%) |
| Total | 124 (100%) | 95 (100%) |

Although the invention has been described with reference to the above examples, it will be understood that modifications and variations are encompassed within the spirit and scope of the invention. Accordingly, the invention is limited only by the following claims.

What is claimed is:

1. A method of isolating or obtaining uncapped nucleotide sequences, the method comprising
contacting a sample containing a population of nucleotide sequences and at least one agent that selectively binds uncapped nucleotide sequences, under conditions that allow the agent to selectively bind an uncapped nucleotide sequence, and
removing the agent and any nucleotide sequence bound thereto from the sample, thereby isolating or obtaining uncapped nucleotide sequences.

2. The method of claim 1, wherein the agent comprises an oligonucleotide, a peptide, or a small organic molecule.

3. The method of claim 1, wherein the agent comprises at least one first member of a specific binding pair.

4. The method of claim 3, wherein removing the agent and any nucleotide sequence bound thereto comprises contacting the sample with at least one second member of the specific binding pair.

5. The method of claim 1, wherein, following removing the agent and any nucleotide sequence bound thereto from the sample, the method further comprises isolating nucleic acid molecules from the sample, thereby obtaining an enriched population of nucleic acid molecules comprising a 5′-cap.

6. The method of claim 5, wherein the nucleic molecules comprising a 5′-cap are ribonucleic acid (RNA) molecules.

7. The method of claim 5, wherein isolating nucleic acid molecules from the sample comprises contacting the sample with at least one polynucleotide that can specifically hybridize to a polyadenosine nucleotide sequence, and isolating nucleic acid molecules that specifically hybridize to the polynucleotide, thereby obtaining an enriched population of nucleic acid molecules comprising 5′-cap and a polyadenosine nucleotide sequence.

8. The method of claim 7, wherein the nucleic acid molecules comprising 5′-cap and a polyadenosine nucleotide sequence comprise full length messenger RNA molecules.

9. An enriched population of nucleic acid molecules obtained by the method of claim 1.

10. A method of obtaining an enriched population of nucleic acid molecules comprising a 5′-cap from a population of nucleotide sequences, the method comprising
contacting a sample containing the population of nucleotide sequences and at least one agent that selectively binds at or near a free 5′-phosphate group of a nucleotide sequence, under conditions that allow the agent to selectively bind at or near a free 5′-phosphate group of a nucleotide sequence, and
removing the agent and any nucleotide sequence bound thereto from the sample, thereby obtaining an enriched population of nucleic acid molecules comprising a 5′-cap.

11. The method of claim 10 wherein the agent comprises an oligonucleotide, a peptide, or a small organic molecule.

12. The method of claim 10, wherein the agent is an oligonucleotide, and the sample further comprises a nucleic acid ligase.

13. The method of claim 10, wherein the nucleic acid molecules comprising a 5′-cap are ribonucleic acid (RNA) molecules.

14. The method of claim 13, wherein the agent comprises an oligonucleotide, a peptide or a small organic molecule.

15. The method of claim 13, wherein the agent is an oligonucleotide comprising a 2′ hydroxyl group or a 3′ hydroxyl group, which can selectively bind a free 5′ phosphate group of the nucleotide sequences.

16. The method of claim 15, wherein the sample further comprises an RNA ligase.

17. The method of claim 16, wherein the RNA ligase is a T4 RNA ligase, E. coli 2′-5′ RNA ligase, or a ribozyme having RNA ligase activity.

18. The method of claim 13, wherein the agent is an oligonucleotide comprising a 3′ hydroxyl group, and wherein the sample further comprises an RNA ligase.

19. The method of claim 18, wherein the RNA ligase is T4 RNA ligase.

20. The method of claim 13, wherein the agent is an oligonucleotide comprising a 2′ hydroxyl group, and wherein the sample further comprises an RNA ligase.

21. The method of claim 20, wherein the RNA ligase is E. coli 2′-5′ RNA ligase.

22. The method of claim 10, wherein removing the agent comprises contacting the sample with at least one moiety that selectively binds the agent.

23. The method of claim 20, wherein the moiety is coupled to a solid support.

24. The method of claim 10, wherein the agent comprises at least one first member of a specific binding pair.

25. The method of claim 24, wherein removing the agent comprises contacting the sample with at least one second member of the specific binding pair, which selectively binds the first member of the specific binding pair.

26. The method of claim 25, wherein the specific binding pair is biotin and avidin, biotin and streptavidin, an antibody specific for an epitope and the epitope, nickel ion and polyhistidine, or glutathione and glutathione S-transferase.

27. The method of claim 24, wherein the agent is an oligonucleotide.

28. The method of claim 27, wherein the oligonucleotide is an oligoribonucleotide.

29. The method of claim 28, wherein the first member of the specific binding pair is biotin.

30. The method of claim 29, wherein removing the agent comprises contacting the sample with avidin or streptavidin.

31. The method of claim 30, wherein the avidin or streptavidin is coupled to a solid support.

32. The method of claim 10, further comprising
contacting the enriched population of nucleic acid molecules with at least one polynucleotide that specifically hybridizes to a polyadenosine nucleotide sequence of a nucleic acid molecule, and
isolating nucleic acid molecules that selectively hybridize to the polynucleotide.

33. The method of claim 32, wherein the nucleic acid molecules that specifically hybridize to the polynucleotide are mRNA molecules.

34. The method of claim 3, further comprising contacting the mRNA molecules with a polypeptide having reverse transcriptase activity and, optionally, with a polypeptide having DNA polymerase activity, thereby producing a single stranded or double stranded cDNA molecule.

35. An enriched population of nucleic acid molecules comprising a 5′-cap prepared by the method of claim 10.

36. The enriched population of claim 35, which comprises an enriched population of full length mRNA molecules.

37. A method of obtaining an enriched population of ribonucleic acid (RNA) molecules comprising a 5′-cap from a population of nucleotide sequences containing RNA molecules comprising a 5′-cap, the method comprising
contacting a sample containing the population of nucleotide sequences and at least one agent that selectively binds at or near a free 5′-phosphate group of a nucleotide sequence, under conditions that allow the agent to bind to a free 5′-phosphate group of a nucleotide sequence; and
removing nucleotide sequences having the agent bound thereto from the sample, thereby obtaining an enriched population of RNA molecules comprising a 5′-cap, thereby obtaining an enriched population of RNA molecules comprising a 5′-cap.

38. The method of claim 37, wherein the agent comprises at least one first member of a specific binding pair.

39. The method of claim 38, wherein removing nucleotide sequences having the agent bound thereto comprises contacting the sample with at least one second member of the specific binding pair, and removing nucleotide sequences having the second member of the specific binding pair bound to the agent.

40. The method of claim 38, wherein the agent comprising at least one first member of a specific binding pair comprises a biotinylated oligoribonucleotide, and wherein the second member of the specific binding pair comprises avidin or streptavidin.

41. The method of claim 40, wherein removing nucleotide sequences having the second member of the specific binding pair bound to the agent comprises contacting the sample with phenol/chloroform, and removing the aqueous fraction, which contains RNA molecules comprising a 5′-cap.

42. The method of claim 40, wherein the avidin or streptavidin is coupled to a solid support, and wherein removing nucleotide sequences having the second member of the specific binding pair bound to the agent comprises contacting the sample with the solid support, and removing the sample from the solid support.

43. A method of obtaining full length messenger ribonucleic acid (mRNA) molecules comprising a 5′-cap and a polyA tail from a population of nucleotide sequences containing full length mRNA molecules, the method comprising
contacting a sample containing the population of nucleotide sequences and at least one agent that selectively binds at or near a free 5′-phosphate group of a nucleotide sequence, under conditions that allow the agent to bind to a free 5′-phosphate group of a nucleotide sequence;
removing nucleotide sequences having the agent bound thereto from the sample, thereby obtaining an enriched population of RNA molecules comprising a 5′-cap;
contacting the enriched population of RNA molecules comprising a 5′-cap with at least one polynucleotide that selectively hybridizes to a polyadenosine nucleotide sequence, under conditions that allow selective hybridization; and
isolating RNA molecules that selectively hybridize to the polynucleotide, thereby obtaining an enriched population of full length mRNA molecules comprising a 5′-cap and a polyA tail.

44. An enriched population of full length mRNA molecules comprising a 5′-cap and a polyA tail produced by the method of claim 43.

45. A method of obtaining an enriched population of full length messenger ribonucleic acid (mRNA) molecules comprising a 5′-cap and a polyA tail from a population of nucleotide sequences containing full length mRNA molecules, the method comprising
contacting a sample containing the population of nucleotide sequences with at least one polynucleotide that selectively hybridizes to a polyadenosine nucleotide sequence, under conditions that allow selective hybridization;
isolating nucleic acid molecules that selectively hybridize to the polynucleotide, thereby obtaining a population of nucleic acid molecules comprising a polyadenosine nucleotide sequence;
contacting the population of nucleic acid molecules comprising a polyadenosine nucleotide sequence, and at least one agent that selectively binds at or near a free 5′-phosphate group of a nucleotide sequence, under conditions that allow the agent to bind to a free 5′-phosphate group of a nucleotide sequence; and
removing nucleotide sequences having the agent bound thereto from the sample, thereby obtaining an enriched population of RNA molecules comprising a 5′-cap;, thereby obtaining an enriched population of full length mRNA molecules comprising a 5′-cap and a polyA tail.

46. An enriched population of full length mRNA molecules comprising a 5′-cap and a polyA tail produced by the method of claim 45.

47. A kit, comprising a carrier means, which can be compartmentalized to receive in close confinement therein one or more containers, which can contain therein one or more components selected from
at least one agent that selectively binds to an uncapped nucleotide sequence;
two or more different agents, each of which selectively binds to an uncapped nucleotide sequence;
at least one agent that selectively binds to an uncapped nucleotide sequence, wherein the agent comprises at least one first member of a specific binding pair;
at least one first member of a specific binding pair;
at least one first member of at least two specific binding pairs, wherein the at least two specific binding pairs can be the same or different;
a reagent for incorporating at least one first member of a specific binding pair into an agent that selectively binds to an uncapped nucleotide sequence;
at least one second member of a specific binding pair, which can be the same or different;
at least one second member of at least two specific binding pairs, which can be the same or different; or
instructions for using the one or more components to isolate an uncapped nucleotide sequence.

48. The kit of claim 47, wherein the at least one agent is an oligoribonucleotide.

49. The kit of claim 48, wherein the oligoribonucleotide comprises at least one first member of a specific binding pair.

50. The kit of claim 49, wherein the first member of the specific binding pair is biotin.


SEQUENCE LISTING (3 sequences)

SEQ ID NO: 1 - RNA, 20 nt, Artificial Sequence
  biotin oligoribonucleotide
gagacgauuuacgauuuacg

SEQ ID NO: 2 - DNA, 16 bp, Artificial Sequence
  M13 reverse primer
aacagctatgaccatg

SEQ ID NO: 3 - DNA, 17 bp, Artificial Sequence
  M13 forward primer
gtaaaacgacggccagt